(19) United States
(12) Patent Application Publication
(10) Pub. No.: US 2026/0130979 A1
(43) Pub. Date: May 14, 2026

(54) MALARIA IMMUNOGEN AND METHODS OF USING SAME

(71) Applicant: UNM RAINFOREST INNOVATIONS, Albuquerque, NM (US)

(72) Inventors: Bryce C. CHACKERIAN, Albuquerque, NM (US); Alexandra FRANCIAN, Albuquerque, NM (US)

(21) Appl. No.: 19/121,222

(22) PCT Filed: Oct. 26, 2023
(86) PCT No.: PCT/US2023/035978

§ 371 Date: Apr. 15, 2025

Related U.S. Application Data
(60) Provisional application No. 63/420,329, filed on Oct. 28, 2022.

(51) Int. Cl. A61K 39/015; A61K 39/00; A61K 39/385; A61P 33/06; A61P 37/04

(52) U.S. Cl. CPC A61K 39/015; A61K 39/0003; A61K 39/385; A61P 33/06; A61P 37/04; A61K 2039/5258; A61K 2039/55561; A61K 2039/627; C12N 2795/18123

(57) ABSTRACT

An immunogen generally includes an immunogenic carrier that includes a virus-like particle (VLP) and an antigenic Anopheles spp. TRIO peptide that includes amino acids VDDLMAKFN (SEQ ID NO:1) or AANLRDKFN (SEQ ID NO:5) linked to the immunogenic carrier. The immunogen can be formulated into a composition such as vaccine. The composition or vaccine may be used to treat a subject having, or at risk of having malaria. The composition or vaccine may be used to treat a subject having Plasmodium falciparum blood stage parasitemia.

### CROSS-REFERENCE TO RELATED APPLICATION

This application claims the benefit of U.S. Provisional Patent Application No. 63/420,329, filed Oct. 28, 2022, which is incorporated herein by reference in its entirety.

### GOVERNMENT FUNDING

This invention was made with government support under AI169739 awarded by the National Institutes of Health. The government has certain rights in the invention.

### SEQUENCE LISTING

This application contains a Sequence Listing electronically submitted via Patent Center to the United States Patent and Trademark Office as an .xml file entitled “0310_000177WO01 ST26 SEQ LISTING.xml” having a size of 6.06 kilobytes and created on Oct. 25, 2023. The information contained in the Sequence Listing is incorporated by reference herein.

### SUMMARY

This disclosure describes, in one aspect, an immunogen that generally includes an immunogenic carrier that includes a virus-like particle (VLP) and an antigenic Anopheles spp. TRIO peptide linked to the immunogenic carrier.

In one or more embodiments, the antigenic Anopheles spp. TRIO peptide includes VDDLMAKFN (SEQ ID NO:1) or a structurally similar variant thereof.

In one or more embodiments, the antigenic Anopheles spp. TRIO peptide includes AANLRDKFN (SEQ ID NO:5) or a structurally similar variant thereof.

In one or more embodiments, the immunogenic carrier is linked to the TRIO peptide through a succinimidyl-6-[β-maleimidopropionamido]hexanoate (SMPH) cross-linker molecule.

In one or more embodiments, the immunogen further includes an antigenic malaria circumsporozoite protein (CSP) peptide or a structurally similar variant thereof. In one or more of these embodiments, the TRIO peptide and the antigenic CSP peptide are displayed on a single VLP.

In another aspect, this disclosure describes a composition that includes any embodiment of an immunogen that includes a virus-like particle (VLP) and an antigenic Anopheles spp. TRIO peptide linked to the immunogenic carrier.

In one or more embodiments, the composition includes a first population of VLPs displaying an antigenic Anopheles TRIO peptide or a structurally similar variant thereof and a second population of VLPs displaying an antigenic CSP peptide or a structurally similar variant thereof.

In one or more embodiments, the composition includes a first population of VLPs displaying an antigenic Anopheles TRIO peptide including SEQ ID NO:1 or a structurally similar variant thereof, and a second population of VLPs displaying an antigenic Anopheles TRIO peptide including SEQ ID NO:5 or a structurally similar variant thereof.

In one or more embodiments, the composition further includes an adjuvant. In one or more of these embodiments, the adjuvant includes a CpG oligonucleotide.

In another aspect, this disclosure describes a nucleic acid that encodes any embodiment of an immunogen that includes a virus-like particle (VLP) and an antigenic Anopheles spp. TRIO peptide linked to the immunogenic carrier.

In another aspect, this disclosure describes an expression vector that includes any embodiment of the nucleic acid summarized above.

In another aspect, this disclosure describes a host cell that includes any embodiment of the expression vector summarized above.

In another aspect, this disclosure describes a vaccine that includes any embodiment of a composition that includes any embodiment of an immunogen that includes a virus-like particle (VLP) and an antigenic Anopheles spp. TRIO peptide linked to the immunogenic carrier.

In another aspect, this disclosure describes a method of treating malaria in an individual. Generally, the method including administering a therapeutically effective amount of a composition to the individual, the composition including an immunogen, the immunogen including an immunogenic carrier and an antigenic Anopheles spp. TRIO peptide linked to the immunogenic carrier.

In one or more embodiments, the antigenic Anopheles spp. TRIO peptide include SEQ ID NO:1 or a structurally similar variant thereof.

In one or more embodiments, the antigenic Anopheles spp. TRIO peptide include SEQ ID NO:5 or a structurally similar variant thereof.

In one or more embodiments, the method further includes administering to the individual at least one additional therapeutic agent for treating malaria.

In one or more embodiments, the immunogen further includes an antigenic malaria circumsporozoite protein (CSP) peptide or a structurally similar variant thereof. In one or more of these embodiments, the TRIO peptide and the antigenic CSP peptide are linked to a single carrier.

In one or more embodiments, the composition further includes a second population of immunogens, the second population of immunogen including a second population of immunogenic carriers and an immunogenic CSP peptide linked to the second population of immunogenic carriers.

In one or more embodiments, the immunogenic carrier includes a Qβ virus-like particle (VLP).

In another aspect, this disclosure describes a method of treating malaria in an individual. Generally, the method includes administering to the individual a therapeutically effective amount of any embodiment of the vaccine summarized above.

In one or more embodiments, the method further includes administering to the individual at least one additional therapeutic agent for treating malaria.

In another aspect, this disclosure describes a method of treating Plasmodium falciparum blood stage parasitemia in an individual. Generally, the method includes administering to the individual a therapeutically effective amount of any embodiment of the vaccine summarized above.

In one or more embodiments, the method further includes administering to the individual at least one additional therapeutic agent for treating Plasmodium falciparum blood stage parasitemia.

In one or more embodiments, the vaccine is administered to the individual before the individual manifests a symptom or clinical sign of Plasmodium falciparum blood stage parasitemia.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Geometric mean anti-AgTRIO peptide endpoint dilution IgG antibody titers in mice immunized twice (at week 0 and week 3) with 5 μg AgTRIO VLPs (closed circles), or a mixture of 5 μg L9 VLPs plus 5 μg AgTRIO VLPs (open circles). Mice were followed for one year following the initial immunization.

FIG. 2. Endpoint dilution IgG titers in mice. (A) Mice were immunized simultaneously with 5 μg L9 VLPs and 5 μg AgTRIO VLPs. (B) Mice were immunized with 5 μg L9 VLPs and 5 μg AsTRIO VLPs. Titers against full-length CSP (gray squares) or AgTRIO/AsTRIO peptide (black circles) were determined by ELISA. Each symbol represents an individual mouse and lines represent geometric mean titers.

FIG. 3. Endpoint dilution IgG titers in immunized mice following challenge. (A) Challenge with full-length AgTRIO. (B) Challenge with AsTRIO. Mice were immunized with AgTRIO VLPs (A), AsTRIO VLPs (B), or, as a control, wild-type Qβ VLPs (A, data shown) and (B, data not shown). Each symbol represents an individual mouse and lines represent geometric mean titers.

FIG. 4. Parasite liver load (as measured by luminescence) in C57BL6 mice (n=5/group) that were immunized with VLPs displaying an Anopheles stephensii TRIO peptide (AsTRIO VLPs) or a mixture of AsTRIO VLPs with L9 VLPs following Anopheles stephensii mosquito challenge. As controls, mice were either not vaccinated (Naïve group) or immunized with control Qβ VLPs. Background liver luminescence was determined using three uninfected mice. A one-tailed Mann-Whitney test was used to statistically compare groups. Background luminescence was determined using three uninfected mice.

### DETAILED DESCRIPTION OF ILLUSTRATIVE EMBODIMENTS

This disclosure describes a virus-like particle (VLP) that displays an antigenic peptide of the salivary protein TRIO from Anopheles mosquitoes. The TRIO-VLP is useful as a vaccine against malaria. This disclosure further describes methods of preparing a TRIO-VLP vaccine and methods of treatment that includes administering a TRIO-VLP to a subject.

Malaria is a significant global public health concern. A disproportionate share of malarial disease and deaths occurs in Africa and is caused by infection with the Plasmodium falciparum parasite. P. falciparum (Pt) infection is initiated when the Anopheles mosquito injects sporozoites into the blood stream of a human host. Sporozoites are transported quickly to the liver where they transiently multiply within hepatocytes, producing merozoites, which then enter the blood stream where they invade red blood cells (RBCs), replicate further, and cause the symptoms and pathology of malaria.

A number of different malaria vaccine strategies have been proposed, including vaccines that target transmission, the erythrocytic stage in which symptoms occur, and the pre-erythrocytic stage. However, it is likely that only vaccines that target the pre-erythrocytic stage can potentially provide complete protection from infection. Sporozoites can reach hepatocytes in less than an hour following infection, limiting the window of time in which immune effectors can act. In addition, a single infected hepatocyte can seed the blood stage of the malaria life cycle. Thus, there is a high barrier for vaccine-mediated protection at the pre-erythrocytic stage; an effective vaccine must elicit sustained, high-titer antibody responses.

Most attempts to develop pre-erythrocytic vaccines have targeted proteins expressed by the Plasmodium falciparum parasite. This disclosure describes a different approach. This disclosure describes compositions, vaccines, and methods that involve targeting a salivary protein of Anopheles mosquitoes. Antibodies that target the highly expressed Anopheles salivary protein TRIO can enhance protection against Plasmodium infection (Dragovic et al., 2018, Cell Host Microbe 23(4):523-535, PMID: 29649443; Chuang et al, 2022, Infect Immun 90(1):e00359-21, PMID: 34724388). The TRIO-specific monoclonal antibody 13F-1 targets a linear epitope with the amino acid sequence VDDLMAKFN (SEQ ID NO:1) near the carboxy-terminus of TRIO. Monoclonal antibody 13F-1 can provide strong protection against Plasmodium berghei infection in a mouse model. This disclosure describes a TRIO-VLP in which the amino acids VDDLMAKFN (SEQ ID NO:1) are displayed by a VLP carrier. The TRIO-VLP could be used as a stand-alone vaccine to treat malaria infection either prophylactically or therapeutically. Alternatively, The TRIO-VLP may be used in combination with other therapeutic agents (e.g., other anti-malaria vaccines) that target the erythrocyte form of the malaria parasite.

### A Virus-Like Particle (VLP)-Based Vaccine Targeting the Mosquito Salivary Protein, TRIO

Multivalent display of antigens on the surface of virus-like particles (VLPs) increases immunogenicity of the antigens. Thus, VLPs can be used to induce strong, durable antibody responses against diverse target antigens. This disclosure describes the use of VLP display to target the salivary protein TRIO from two mosquito species, Anopheles gambiae (Ag) and Anopheles stephensii (As), that transmit malaria. TRIO enhances malaria infection and modulates the host immune response. A peptide representing the 13F-1 epitope of AgTRIO was synthesized to contain a -GGGC (SEQ ID NO:3) linker sequence (VDDLMAKFNGGGC; SEQ ID NO:2) to allow the antigenic AgTRIO peptide VDDLMAKFN (SEQ ID NO:1) to be conjugated to Qβ bacteriophage VLPs. Similarly, a peptide representing the analogous epitope from AsTRIO (AANLRDKFN; SEQ ID NO:5) with a -GGGC (SEQ ID NO:3) linker sequence was synthesized to produce (AANLRDKFNGGGC; SEQ ID NO:6). Briefly, the amine-reactive arm of the crosslinker succinimidyl 6-((beta-maleimidopropionamido)hexanoate) (SMPH) was linked to surface-exposed lysines on Qβ VLPs by reacting the VLPs with SMPH at a 1:10 molar ratio. Qβ-SMPH conjugates were purified by centrifugation. Qβ-SMPH was linked to the TRIO peptides by virtue of an exposed sulfhydryl residue on the C-terminal cysteine residue of the peptide. Qβ-SMPH was reacted with the TRIO peptide at a 1:10 molar ratio and Qβ-TRIO conjugated particles (referred to as TRIO VLPs) were purified by centrifugation. The extent of modification of VLPs was assessed by polyacrylamide gel electrophoresis.

To assess immunogenicity of TRIO VLPs, mice (n=5) were given two intramuscular injections of 5 μg AgTRIO VLPs or AsTRIO VLPs at a three-week interval. In addition, to assess compatibility with a pre-erythrocytic vaccine, groups of mice were also immunized with a combination vaccine containing either 5 μg of AgTRIO VLPs or AsTRIO VLPs, plus 5 μg of L9 VLPs (U.S. Patent Application Publication No. 2023/0102159 A1). L9 VLPs elicit antibody responses against a particularly vulnerable epitope in the Plasmodium falciparum circumsporozoite protein (CSP) and are effective against blood-stage malaria in an experimental mouse malaria challenge model (U.S. Patent Application Publication No. 2023/0102159 A1; Jelinková et al., 2022, npj Vaccines 7:34, PMID: 35260593). Sera were collected from immunized mice and antibody responses against the AgTRIO peptide, AsTRIO peptide, full-length AgTRIO, full-length AsTRIO, and full-length CSP were measured by ELISA.

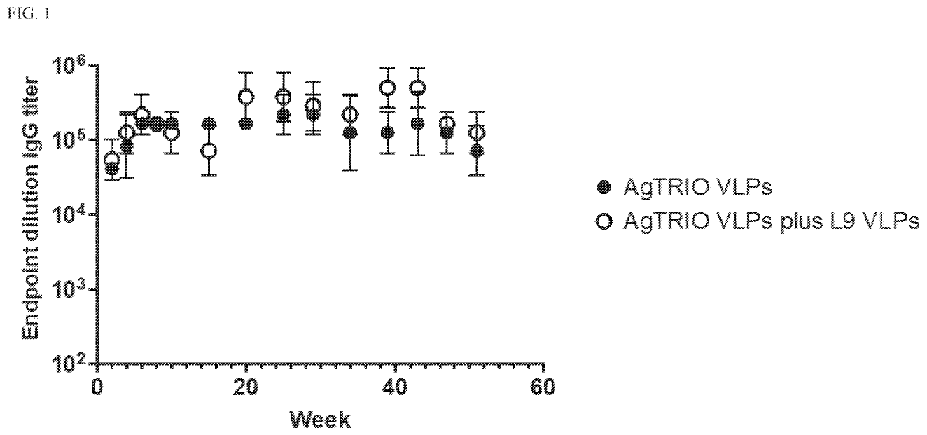

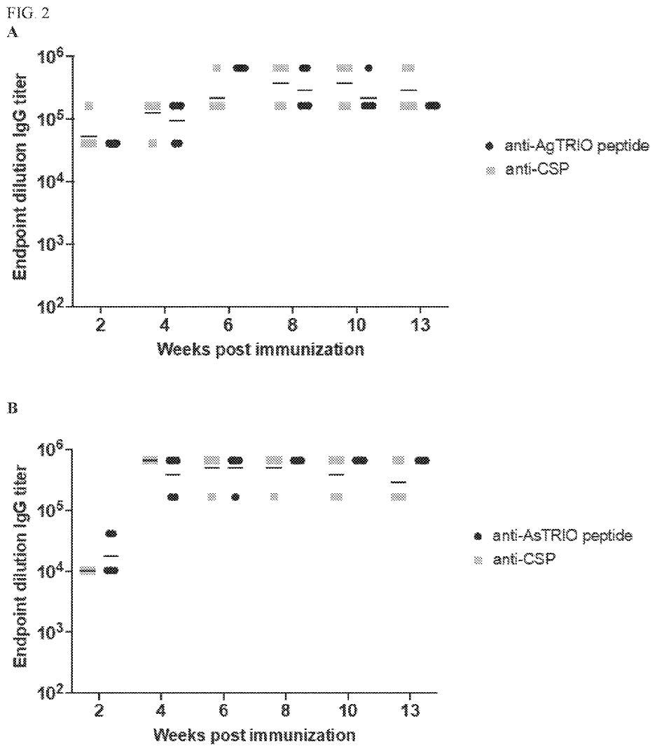

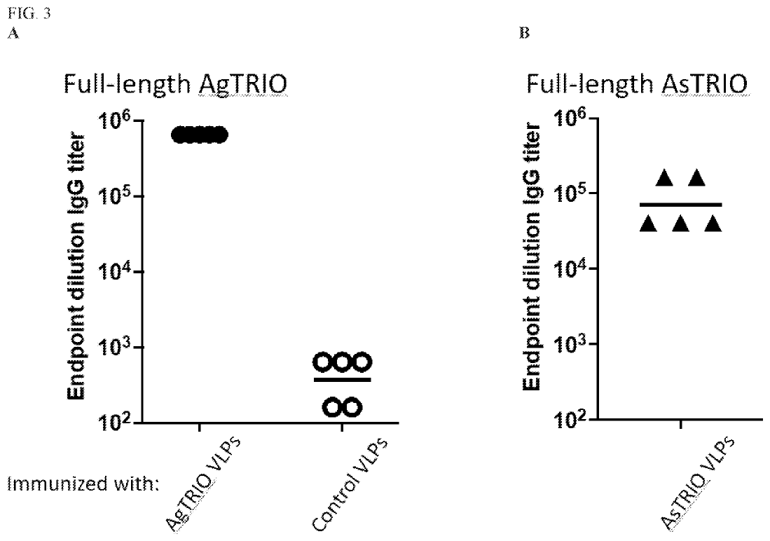

FIG. 1 shows the endpoint dilution IgG antibody titers using sera from mice immunized with either AgTRIO VLPs alone or a combination of AgTRIO VLPs plus L9 VLPs, obtained at various timepoints up to a year after immunization. Both groups of mice elicited high antibody titers to the AgTRIO peptide, regardless of whether the L9 VLPs were included in the vaccine preparation. FIG. 2 shows that mice immunized with combination vaccines consisting of L9 VLPs plus AgTRIO VLPs (panel A) or L9 VLPs plus AsTRIO VLPs (panel B) elicit strong antibody responses against both the TRIO peptide and CSP protein. FIG. 3 shows that antibodies elicited by TRIO VLP vaccines also bind strongly to full-length AgTRIO protein (panel A) or AsTRIO protein (panel B). Thus, this disclosure describes the development and characterization of a bacteriophage VLP-based immunogens and/or vaccines targeting amino acid residues VDDLMAKFN (SEQ ID NO:1) and AANLRDKFN (SEQ ID NO:5) of the Anopheles salivary protein TRIO that elicit strong antibody responses that bind to the target peptides as well as full length TRIO protein.

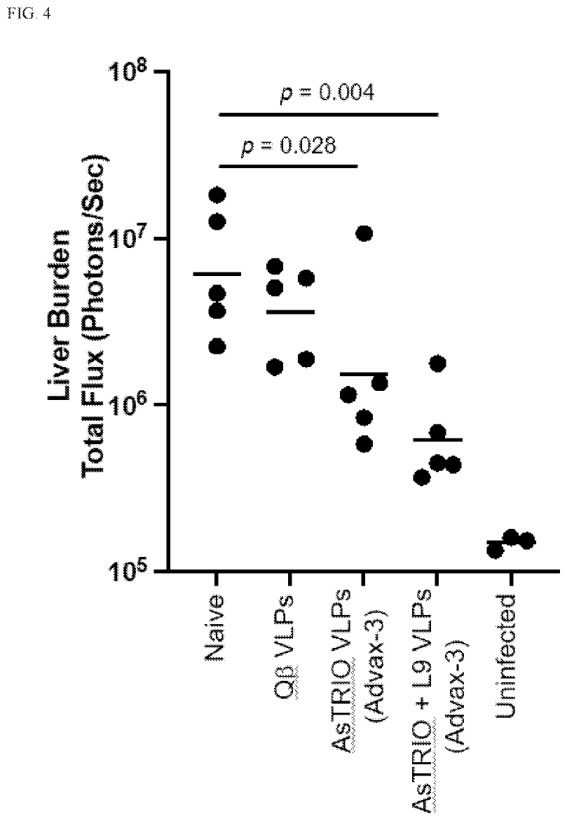

To test whether TRIO VLPs could confer protection from malaria challenge, C57Bl/6 mice were vaccinated with AsTRIO VLPs, a mixture of AsTRIO VLPs and L9 VLPs, or, as a negative control, wild-type Qß VLPs and then challenged with malaria-infected mosquitoes. Test vaccines were adjuvanted with Advax-3, which is a mixture of CpG55.2 oligonucleotide (a TLR9 agonist) with aluminum hydroxide. After three immunizations, mice were exposed to mosquitoes infected with luciferase-reporter containing transgenic P. berghei (Pb) engineered to express full-length PfCSP in place of PbCSP (Pb-PfCSP-Luc). Forty-two hours after challenge, liver parasite loads were measured using an intravital imaging system. As shown in FIG. 4, mice immunized with AsTRIO VLPs and the mixture of AsTRIO VLPs and L9 VLPs had significantly lower liver parasite loads than unvaccinated (naïve) controls. VLPs displaying an AsTRIO peptide reduced parasite liver burden by ˜75%. Co-administration of L9 VLPs with AsTRIO VLPs further reduced liver burden (by ˜90%).

### VLP Display

Many viral structural proteins have an intrinsic ability to self-assemble into virus-like particles (VLPs), which structurally resemble the virus from which they were derived but, because they lack viral genomes, they are absolutely noninfectious. VLPs not only can serve as stand-alone vaccines, but because their particulate nature and multivalent structure provoke strong immune responses, they can be used as platforms to enhance the immunogenicity of heterologous antigenic targets. For example, when short immunogenic peptides are displayed in a highly repetitive, multivalent fashion on VLPs, peptide-specific B cells are strongly activated, leading to high-titer, long-lasting antibody responses. VLPs derived from diverse virus types can serve as effective platforms for antigen display. The immunogens described herein are based on VLPs derived from a family of related single-stranded RNA bacteriophages, including MS2, PP7, AP205, and Qß. These VLPs can be produced by expressing a single viral structural protein, called coat, from a plasmid in a bacterium. Peptides may be displayed on a VLP by bioconjugation techniques using cross linker molecules. In one or more embodiments, a peptide may be displayed on a VLP by conjugating the peptide to the VLP through a succinimidyl-6-[β-maleimidopropionamido]hexanoate (SMPH) cross-linker molecule. This technique results in VLPs that display target peptides at high valency, usually 180-360 peptides per VLP, and confers strong immunogenicity to displayed immunogenic peptides.

### Construction and Antigenicity of TRIO VLPs

Qß VLPs that multivalently display the antigenic nine-amino-acid 13F-1 epitope (SEQ ID NO: 1) of A. gambiae salivary protein TRIO (AgTRIO) were constructed by chemically conjugating the antigenic TRIO peptide to the surface of VLP using a bifunctional crosslinker. Qß VLPs that multivalently display the antigenic nine-amino-acid epitope (SEQ ID NO: 5) of A. stephensii salivary protein TRIO (AsTRIO) were constructed similarly.

While described below in the context of an exemplary embodiment in which the VLP platform used to present the antigenic TRIO peptide is a Qβ VLP, the compositions and methods described herein can involve the use of any suitable VLP platform. Thus, as noted above, VLPs that present an antigenic TRIO peptide can be derived from any one of a family of related single-stranded RNA bacteriophages including, but not limited to, MS2, PP7, AP205, or Qß.

The VLP-based immunogen thus includes an antigenic TRIO peptide (also referred to herein as a “a TRIO-targeting peptide”) such as, for example, the amino acids of SEQ ID NO:1, SEQ ID NO: 5, or a structurally similar peptide. Further, the immunogen can include a VLP that displays more than one population of antigenic peptides—e.g., a first population of antigenic TRIO peptides that includes the amino acids of SEQ ID NO:1, SEQ ID NO:5 (or a structurally similar variant thereof) and a second population of antigenic peptides—e.g., an antigenic CSP peptide including, but not limited to, antigenic CSP peptides described in International Patent Application No. PCT/2020/043375 (international Publication No. WO 2021/016509 A1) or U.S. Patent Application Publication No. 2023/0102159 A1, or a peptide structurally similar to any of the foregoing. Thus, the immunogen can be designed to display one, two, three, four, five, six, or more antigenic peptides.

In another aspect, an immunogenic composition may include more than one population of VLPs. For example, an immunogenic composition can include a first population of VLPs displaying the antigenic TRIO peptide including the amino acids of SEQ ID NO:1 or SEQ ID NO:5 (or a structurally similar variant thereof) and a second population of VLPs displaying a second antigenic peptide. Exemplary second antigenic peptides include, but are not limited to, an antigenic CSP peptide including, but not limited to, the amino acids of SEQ ID NO:1, the amino acids of SEQ ID NO:5, an antigenic CSP peptide described in International Patent Application No. PCT/2020/043375 (International Publication No. WO 2021/016509 A1), an antigenic CSP peptide described in U.S. Patent Application Publication No. 2023/0102159 A1, or a peptide structurally similar to any of the foregoing.

As used herein, a peptide or variant of a peptide is “structurally similar” to a reference peptide if the amino acid sequence of the peptide possesses a specified amount of identity compared to the reference peptide. Structural similarity of two peptides can be determined by aligning the residues of the two peptides (for example, a candidate polypeptide and SEQ ID NO:1) to optimize the number of identical amino acids along the lengths of their sequences, gaps in either or both sequences are permitted in making the alignment in order to optimize the number of identical amino acids, although the amino acids in each sequence must nonetheless remain in their proper order. A candidate peptide is the peptide being compared to the reference peptide (e.g., SEQ ID NO:1 or SEQ ID NO:5). A candidate peptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, peptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

An antigenic TRIO peptide can include amino acids in addition to the amino acids of SEQ ID NO:1 or SEQ ID NO:5, so long as the additional amino acids do not eliminate immunogenicity toward TRIO. For example, an antigenic TRIO peptide may have a linker region containing the amino acids GGGC (SEQ ID NO: 2) to generate the peptide of SEQ ID NO:3 or SEQ ID NO:6.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also includes the presence of conservative substitutions. A conservative substitution for an amino acid in an immunogenic peptide as described herein may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the peptide are also contemplated.

In one or more embodiments, a TRIO-targeting peptide as described herein can include a peptide with at least 66%, at least 77%, or at least 88% sequence similarity to amino acids SEQ ID NO: 1. That is, a TRIO-targeting polypeptide can include a total of no more than three, no more than two, or no more than one amino acid deletions and non-conservative amino acid substitutions compared to SEQ ID NO:1.

In one or more embodiments, a TRIO-targeting peptide as described herein can include a peptide with at least at least 66%, at least 77%, or at least 88% sequence identity to SEQ ID NO:1. That is, a TRIO-targeting polypeptide can include a total of no more than three, no more than two, or no more than one amino acid deletions and amino acid substitutions compared to SEQ ID NO:1.

In one or more embodiments, a TRIO-targeting peptide as described herein can include a peptide with at least 66%, at least 77%, or at least 88% sequence similarity to amino acids SEQ ID NO:5. That is, a TRIO-targeting polypeptide can include a total of no more than three, no more than two, or no more than one amino acid deletions and non-conservative amino acid substitutions compared to SEQ ID NO:5.

In one or more embodiments, a TRIO-targeting peptide as described herein can include a peptide with at least at least 66%, at least 77%, or at least 88% sequence identity to SEQ ID NO:5. That is, a TRIO-targeting polypeptide can include a total of no more than three, no more than two, or no more than one amino acid deletions and amino acid substitutions compared to SEQ ID NO:5.

In one or more embodiments, a TRIO-targeting peptide as described herein can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel columns. Such gene modification techniques and suitable additional sequences are well known in the molecular biology arts.

The virus-like particle (VLP) can include any particle that includes viral protein assembled to structurally resemble the virus from which they are derived, but lack enough of the viral genome so that they are non-replicative and, therefore, noninfectious. A VLP may, therefore, include at least some of the viral genome, but the viral genome is genetically modified so that the viral genes responsible for infectivity and replication are inactivated. Exemplary VLPs include, but are not limited to, VLPs of Qβ, MS2, PP7, AP205, or other bacteriophage coat proteins, the capsid and core proteins of Hepatitis B virus, measles virus, Sindbis virus, rotavirus, foot-and-mouth-disease virus, Norwalk virus, the retroviral GAG protein, the retrotransposon Ty protein pl, the surface protein of Hepatitis B virus, human papilloma virus, human polyoma virus, RNA phages, Ty, frphage, GA-phage, AP 205-phage and, in particular, Qβ-phage, Cowpea chlorotic mottle virus, cowpea mosaic virus, human papilloma viruses (HPV), bovine papilloma viruses, porcine parvovirus, parvoviruses such as B19, porcine (PPV) and canine (CPV) parvovirues, caliciviruses (e.g. Norwalk virus, rabbit hemorrhagic disease virus [RHDV]), animal hepadnavirus core Antigen VLPs, filamentous/rod-shaped plant viruses, including but not limited to Tobacco Mosaic Virus (TMV), Potato Virus X (PVX), Papaya Mosaic Virus (PapMV), Alfalfa Mosaic Virus (AIMV), and Johnson Grass Mosaic Virus (JGMV), insect viruses such as flock house virus (FHV) and tetraviruses, polyomaviruses such as Murine Polyomavirus (MPyV), Murine Pneumotropic Virus (MPtV), BK virus (BKV), and JC virus (JCV).

The antigenic TRIO peptides may be coupled to immunogenic carriers via chemical conjugation or by expression of genetically engineered fusion partners. The coupling does not necessarily need to be direct, but can occur through linker sequences. More generally, in the case that antigenic peptides either fused, conjugated, or otherwise attached to an immunogenic carrier, spacer sequence, or linker sequence are typically added at one or both ends of the antigenic peptides. Such linker sequences generally comprise sequences recognized by the proteasome, proteases of the endosomes or other vesicular compartment of the cell.

In one embodiment, the antigenic TRIO peptide may be displayed as fusion protein with a subunit of the immunogenic carrier. Fusion of the peptide can be effected by inserting the TRIO antigenic peptide amino acid sequence into the immunogenic carrier primary sequence, or by fusion to either the N-terminus or C-terminus of the immunogenic carrier.

When the immunogenic carrier is a VLP, the chimeric antigenic peptide-VLP subunit can be capable of self-assembly into a VLP. VLP displaying epitopes fused to their subunits are also herein referred to as chimeric VLPs. For example, European Patent No. EP 0421635 B1 describes the use of chimeric hepadnavirus core antigen particles to present foreign peptide sequences in a virus-like particle.

Flanking amino acid residues may be added to either end of the sequence of the antigenic peptide to be fused to either end of the sequence of the subunit of a VLP, or for internal insertion of such peptide sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In one or more embodiments, the immunogenic carrier is a VLP of a RNA phage, preferably Qβ. The major coat proteins of RNA phages spontaneously assemble into VLPs upon expression in bacteria such as, for example, E. coli. Fusion protein constructs wherein antigenic peptides have been fused to the C-terminus of a truncated form of the A1 protein of Qβ, or inserted within the A1 protein have been described (Kozlovska et al., 1996. Intervirology 39: 9-15). Assembly of Qβ particles displaying the fused epitopes typically involves the presence of both the A1 protein-antigen fusion and the wild type coat protein to form a mosaic particle. However, embodiments involving VLPs, and in particular the VLPs of the RNA phage Qβ coat protein, that are exclusively composed of VLP subunits having an antigenic peptide fused thereto, are contemplated.

The production of mosaic particles may be effected in a number of ways. In one exemplary approach, efficient display of the fused epitope on the VLPs is mediated by the expression of the plasmid encoding the Qβ A1 protein fusion having a UGA stop codon between the coat protein and the coat protein extension in an E. coli strain harboring a plasmid encoding a cloned UGA suppressor tRNA, which leads to translation of the UGA codon into Trp (pISM3001 plasmid). In a second exemplary approach, the coat protein gene stop codon is modified into UAA, and a second plasmid expressing the A1 protein-antigen fusion is co-transformed. The second plasmid encodes a different antibiotic resistance and the origin of replication is compatible with the first plasmid. In a third exemplary approach. Qβ coat protein and the A1 protein-antigen fusion are encoded in a bicistronic manner, operatively linked to a promoter such as the Trp promoter.

Further VLPs suitable for fusion of antigens or antigenic determinants are described in, for example, International Patent Application No. PCT/IB2002/004132 (International Publication No. WO 03/024481 A2) and include bacteriophage fr, RNA phase MS-2, capsid protein of papillomavirus, retrotransposon Ty, yeast and also Retrovirus-like-particles, HIV2 Gag, Cowpea Mosaic Virus, parvovirus VP2 VLP, hBsAg (U.S. Pat. No. 4,722,840). Examples of chimeric VLPs suitable for use as the immunogenic carrier include those described in Kozlovska et al., 1996, Intervirology 39.9-15. Further examples of VLPs suitable for use as the immunogenic carrier include, but are not limited to, HPV-1, HPV-6, HPV-11, HPV-16, HPV-18. HPV-33, HPV-45, CRPV, COPV, HIV GAG, Tobacco Mosaic Virus, Virus-like particles of SV-40, Polyomavirus, Adenovirus, Herpes Simplex Virus, Rotavirus, and Norwalk virus.

In a preferred embodiment, a vaccine construct containing the TRIO peptide containing SEQ ID NO:1 is synthesized by conjugating the peptide to Qβ bacteriophage VLPs using a bifunctional cross-linker (SMPH). The TRIO peptide can be modified to include a linker peptide to the C-terminus (e.g., a GGGC linker sequence; SEQ ID NO:2 and SEQ ID NO:6) or the N-terminus (e.g., a CGGG linker sequence; SEQ ID NO:4). The SMPH cross-linker conjugates free amines on the surface of the Qβ VLPs to the cysteine residue of the linker peptide. In this synthesis methodology, the Qβ VLP is purified from free, unconjugated crosslinker, and then reacted with the TRIO peptide at a molar ratio of about 10 peptides:1 VLP

For any recombinantly expressed antigenic TRIO peptide described herein (whether or not coupled to an immunogenic carrier), this disclosure describes the nucleic acid that encodes the peptide or protein, as is an expression vector comprising the nucleic acid, and a host cell containing the expression vector (autonomously or chromosomally inserted). This disclosure further describes a method of recombinantly producing the peptide or protein by expressing it in a host cell with or without further isolating the immunogen.

Thus, this disclosure describes an isolated nucleic acid sequence that encodes any embodiment of an antigenic TRIO peptide described herein. In one or more embodiments, the isolated nucleic acid encodes the antigenic peptide of SEQ ID NO: 1, the antigenic peptide of SEQ ID NO:5, or a structurally similar variant of either SEQ NO:1 or SEQ ID NO:5. Given the amino acid sequence of any immunogenic TRIO peptide, a person of ordinary skill in the art can determine the full scope of polynucleotides that encode that amino acid sequence using conventional, routine methods.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be introduced—i.e., transfected—into cells. When RNA is used to transfect cells, the RNA may be modified by stabilizing modifications, capping, or polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, which relies on agarose gel electrophoresis, polyacrylamide gel electrophoresis, or high-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

This disclosure also describes a host cell including any of the isolated nucleic acid sequences and/or antigenic peptides described herein. Thus, this disclosure encompasses translation of a nucleic acid (e.g., an mRNA) by a host cell to produce an immunogenic TRIO peptide and/or a VLP that displays an immunogenic TRIO peptide.

The nucleic acid constructs of the present invention may be introduced into a host cell to be altered, thus allowing expression of the TRIO peptide and/or TRIO VLP within the cell, thereby generating a genetically engineered cell. A variety of methods are known in the art and suitable for introducing a nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc, Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the protein encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

In one or more embodiments, the antigenic TRIO peptide can be chemically coupled to the immunogenic carrier using techniques well known in the art. Conjugation can occur to allow free movement of peptides via single point conjugation (e.g., either N-terminal or C-terminal point) or as a locked down structure where both ends of peptides are conjugated to either an immunogenic carrier protein or to a scaffold structure such as a VLP. Conjugation occurs via conjugation chemistry known to those skilled in the art such as via cysteine residues, lysine residues, or another carboxy moiety. Thus, for example, for direct covalent coupling, it is possible to use a carbodiimide, glutaraldehyde, or N-[y-maleimidobutyryloxy]succinimide ester, using common commercially available hetero-bifunctional linkers such as 1-cyano-4-dimethylaminopyridinium tetrafluoroborate (CDAP) or succinimidyl 3-(2-pyridyldithio)propionate (SPDP).

Examples of conjugation of peptides, particularly cyclized peptides, to a protein carrier via acylhydrazine peptide derivatives are described in, for example, International Patent Application No. PCT/EP2003/004551 (International Publication No. WO 2003/092714 A1). After the coupling reaction, the immunogen can easily be isolated and purified using, for example, a dialysis method, a high performance liquid chromatography method, a gel filtration method, a fractionation method, etc. Peptides terminating with a cysteine residue (preferably with a linker outside the cyclized region) may be conveniently conjugated to a carrier protein via maleimide chemistry.

Several antigenic peptides, either having an identical amino acid sequence or different amino acid sequences, may be coupled to a single VLP particle, leading preferably to a repetitive and ordered structure presenting several antigenic determinants in an oriented manner as described in International Patent Applications PCT/IB1999/001925 (International Publication No. WO 2000/032227), PCT/IB2002/004132 (International Publication No. WO 2003/024481), PCT/IB2002/000166 (International Publication No. WO 2002/056905), and PCT/EP2003/007572 (International Publication No. WO 2004/007538). Thus, the antigenic peptide displayed by one VLP subunit in a VLP may the same or different than the antigenic peptide displayed by a second VLP subunit in the same VLP. In other embodiments, one or several antigen molecules can be attached to one VLP subunit. A specific feature of the VLP of the coat protein of RNA phages, and in particular of the Qβ coat protein VLP, is thus the possibility to couple several antigens per subunit. This allows for the generation of a dense antigen array.

Another feature of VLPs derived from RNA phage is their high expression yield in bacteria that allows production of large quantities of material at affordable cost. Moreover, the use of the VLPs as carriers allows the formation of robust antigen arrays and conjugates, respectively, with variable antigen density. In particular, the use of VLPs of RNA phages, and in particular the use of the VLP of RNA phage Qβ coat protein, allows a very high antigen density to be achieved.

### Compositions and Methods of Treatment

The TRIO-targeting VLP may be used to treat a subject having, or at risk of having, a condition characterized, at least in part, by being spread by Anopheles spp. mosquitoes whose saliva includes TRIO. Such conditions include, but are not limited to, malaria or Plasmodium falciparum blood stage parasitemia.

As used herein, “treat” or variations thereof refer to reducing, limiting progression, ameliorating, or resolving, to any extent, the symptoms or signs related to a condition. A “sign” or “clinical sign” refers to an objective physical finding relating to a particular condition capable of being found by one other than the patient. A “symptom” refers to any subjective evidence of disease or of a patient's condition.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Typically, the TRIO-targeted VLP will be used prophylactically to induce pre-erythrocytic immunity, thereby reducing the likelihood that the malaria parasite reaches the liver.

Treatment that is prophylactic—e.g., initiated before a subject manifests a symptom or clinical sign of the condition such as, for example, while a tumor remains subclinical—is referred to herein as treatment of a subject that is “at risk” of having the condition. As used herein, the term “at risk” refers to a subject that may or may not actually possess the described risk. Thus, for example, a subject “at risk” of developing a condition is a subject possessing one or more risk factors associated with the condition such as, for example, genetic predisposition, ancestry, age, sex, geographical location, lifestyle, or medical history. Thus, the TRIO-targeted VLP may be administered before a subject manifests a symptom or clinical sign of malaria. In one or more embodiments, the TRIO-targeted VLP may be administered before a subject travels to a geographical location where malaria may be prevalent.

Accordingly, a composition can be administered before, during, or after the subject first exhibits a symptom or clinical sign of the condition (e.g., malaria or Plasmodium falciparum blood stage parasitemia). Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a condition characterized, at least in part, by transmission by Anopheles spp. mosquitoes whose saliva includes TRIO. In this aspect, an “effective amount” is an amount effective to reduce, limit progression, ameliorate, or resolve, to any extent, a symptom or clinical sign related to the condition.

Thus, the TRIO VLP described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the TRIO VLP without causing any undesirable biological effects or interacting in a deleterious manner with any of the other components of the pharmaceutical composition in which it is contained.

The TRIO VLP may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a TRIO VLP may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the TRIO VLP into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of TRIO VLP administered can vary depending on various factors including, but not limited to, the cancer being treated, the weight, physical condition, and/or age of the subject, and/or the route of administration. Thus, the absolute weight of TRIO VLP included in a given unit dosage form can vary widely, and depends upon factors such as the species, age, weight, and physical condition of the subject, and/or the method of administration. Accordingly, it is not practical to set forth generally the amount that constitutes an amount of TRIO VLP effective for all possible applications. Those of ordinary skill in the art, however, can readily determine the appropriate amount with due consideration of such factors.

In one or more embodiments, the method can include administering sufficient TRIO VLP to provide a dose of, for example, from about 50 ng/kg to about 1 mg/kg to the subject, although in one or more embodiments the methods may be performed by administering TRIO VLP in a dose outside this range.

In one or more embodiments, the method includes administering sufficient TRIO VLP to provide a minimum dose of at least 50 ng/kg such as, for example, at least 100 ng/kg, at least 200 ng/kg, at least 300 ng/kg, at least 400 ng/kg, at least 500 ng/kg, at least 600 ng/kg, at least 700 ng/kg, at least 800 ng/kg, at least 900 ng/kg, at least 1 μg/kg, at least 2 μg/kg, at least 5 μg/kg, at least 10 μg/kg, at least 20 μg/kg, at least 50 μg/kg, at least 100 μg/kg, at least 200 μg/kg, or at least 500 μg/kg.

In one or more embodiments, the method includes administering sufficient TRIO VLP to provide a maximum dose of no more than 1 mg/kg, no more than 500 μg/kg, no more than 250 μg/kg, no more than 200 μg/kg, no more than 150 μg/kg, no more than 100 μg/kg, no more than 50 μg/kg, no more than 25 μg/kg, no more than 10 μg/kg, no more than 5 μg/kg, no more than 2 μg/kg, no more than 1 μg/kg, no more than 800 ng/kg, no more than 600 ng/kg, no more than 500 ng/kg, no more than 400 ng/kg, no more than 300 ng/kg, no more than 250 ng/kg, no more than 150 ng/kg, no more than 100 ng/kg, no more than 50 ng/kg, or no more than 25 ng/kg.

In one or more embodiments, the method includes administering sufficient TRIO VLP to provide that falls within a range having as endpoints any minimum dose listed above and any maximum dose listed above that is greater than the minimum does. For example, in one or more embodiments, the method can includes administering sufficient TRIO VLP to provide a dose of from 200 ng/kg to about 10 μg/kg to the subject, for example, a dose of from about 700 ng/kg to about 5 μg/kg.

In one or more embodiments, TRIO VLP may be administered, for example, from a single dose to multiple doses per week, although in one or more embodiments the method can be performed by administering TRIO VLP at a frequency outside this range. When multiple doses are used within a certain period, the amount of each dose may be the same or different. For example, a dose of 1 mg/kg per day may be administered as a single dose of 1 mg/kg, two 0.5 mg/kg doses, or as a first dose of 0.75 mg/kg followed by a second dose of 0.25 mg/kg. Also, when multiple doses are used within a certain period, the interval between doses may be the same or be different.

In certain embodiments, TRIO VLP may be administered at minimum frequency of at least once per year such as, for example, at least once every six months, at least once every four months, at least once every three months, at least once every two months, at least once per month, or at least once every two weeks.

In certain embodiments, TRIO VLP may be administered at maximum frequency of no more than once per week such as, for example, no more than once every two weeks, no more than once per month, no more than once every two months, no more than once every three months, no more than once every six months, or once per year.

In one or more embodiments, TRIO VLP may be administered at a frequency defined by a range having as endpoints any minimum frequency listed above and any maximum frequency listed above that is more frequent than the minimum frequency.

The duration of administration of an antigenic TRIO VLP described herein, e.g., the period of time over which an antigenic TRIO VLP is administered, can vary, depending on any of a variety of factors, e.g., patient response, etc. For example, an antigenic TRIO VLP can be administered over a period of time ranging from about one day to about one week, from about two weeks to about four weeks, from about one month to about two months, from about two months to about four months, from about four months to about six months, from about six months to about eight months, from about eight months to about one year, from about one year to about two years, or from about two years to about four years, or more. In one or more embodiments, the TRIO VLP may be administered as a once off treatment. In other embodiments, the TRIO VLP may be administered for the life of the subject. In certain embodiments, the TRIO VLP may be administered may be administered monthly (or every four weeks) until effective.

In one or more embodiments, the TRIO VLP may be administered at an initial frequency for an initial period and then administered at a lower frequency thereafter. For example, a dosing regimen may include administering three doses of the TRIO VLP at a frequency of once per month (i.e., an initial dose followed by a second dose one month after the initial dose) followed by an additional dose six months after the initial dose.

When a TRIO VLP composition is used for prophylactic treatment, it may be generally administered for priming and/or boosting doses Boosting doses, when administered, are adequately spaced (e.g., yearly) to boost the level of circulating antibody that has fallen below a desired level. Boosting doses may include an antigenic TRIO peptide either with or in the absence of the original immunogenic carrier. A booster composition may include an alternative immunogenic carrier or may be in the absence of any carrier. Moreover, a booster composition may be formulated either with or without adjuvant.

In some cases, the method can further include administering to the subject an additional therapeutic agent effective for treating the condition (e.g., malaria). For example, therapy involving the TRIO VLP may be combined with conventional therapies for malaria. As another example, the TRIO VLP can increase efficacy of any pre-erythrocytic malaria treatment (e.g., a pre-erythrocytic malaria vaccine) by, for example, decreasing the efficiency of transmission. Thus, the TRIO VLPs may be used for treatment with a pre-erythrocytic malaria treatment including, but not limited to, malaria vaccines such as an L9 VLP vaccine, an RTS,S vaccine (e.g., the RTS,S/AS01 vaccine), an R21 vaccine, or a PfSPZ vaccine.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### Examples

The present invention is illustrated by the following examples. It is to be understood that the particular examples, materials, amounts, and procedures are to be interpreted broadly in accordance with the scope and spirit of the invention as set forth herein.

### Production and Characterization of VLP-Based Vaccines

TRIO VLPs were produced using similar techniques as described in (Jelinková et al., 2022, npj Vaccines 6, 13). The nine-amino-acid AgTRIO 13F-1 epitope peptide VDDLMAKFN (SEQ ID NO: 1) was synthesized (GenScript Biotech Corp., Piscataway, NJ) and modified to contain a C-terminal GGGC (SEQ ID NO:2) linker sequence to produce the antigenic TRIO peptide having the amino acid sequence VDDLMAKFNGGGC (SEQ ID NO:3), which was conjugated directly to surface lysines on Qβ bacteriophage VLPs using the bidirectional crosslinker succinimidyl 6-[(beta-maleimidopropionamido) hexanoate](SMPH; Thermo Fisher Scientific Inc., Waltham, MA) as previously described (Tumban et al. PLOS ONE 6, e23310 (2011)). A similar approach was used to display the nine-amino-acid AsTRIO peptide AANLRDKFN (SEQ ID NO:5) on VLPs.

### Mouse Immunization Studies

For the initial evaluation of immunogenicity, groups of 4-5-week-old female Balb/c mice (Jackson ImmunoResearch Laboratories, Inc., West Grove, PA) were immunized intramuscularly with 5 μg of TRIO VLPs at a three-week interval. Separately, a group of mice was immunized intramuscularly with a combination vaccine containing 5 μg of TRIO VLPs plus 5 μg L9 VLPs. The L9 VLPs elicit antibody responses against a particularly vulnerable epitope in the Plasmodium falciparum circumsporozoite protein (CSP) induce immunity against blood-stage malaria in experimental mouse malaria challenge model (Jelinková et al., 2022, npj Vaccines 7:34, PMID: 35260593).

### Quantitating Antibody Responses

Serum antibodies against full-length CSP were detected by ELISA using recombinant CSP expressed in Plasmodium falciparum (Noe et al. PLoS One 9, e107764 (2014)) (Leidos, Inc., Reston, VA) as the coating antigen, as described previously (Jelinková et al., 2022, npj Vaccines 7:34, PMID: 35260593) or with recombinant AgTRIO or AsTRIO expressed in E. coli.

### Mouse Pb-PfCSP-Luc Sporozoite Mosquito Challenge

Groups of mice (n=5) were vaccinated three times at three-week intervals with 5 μg of AsTRIO VLPs, a mixture of 5 μg AsTRIO VLPs plus L9 VLPs, or, as a control, unconjugated Qß VLPs. As an additional control a group of mice were not vaccinated. Mice were challenged directly by using infected mosquitos four weeks following their final vaccination. Anopheles stephensi mosquitos were infected by blood-feeding on Pb-PfCSP-Luc infected mice. Prior to challenge, mice were anesthetized with 2% tribromoethanol, and then exposed to six mosquitos for a blood meal for 10 minutes. Following feeding, the number of mosquitos positive for a blood meal was determined. Liver luminescence was assessed 42 hours post-challenge by intraperitoneally injecting anesthetized mice with 100 μL d-luciferin (30 mg/mL) and then determining liver luminescence using an IVIS Spectrum Imaging System (PerkinElmer, Waltham, MA).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### SEQUENCE LISTING FREE TEXT

AgTRIO antigenic epitope
SEQ ID NO: 1
VDDLMAKEN
linker
SEQ ID NO: 2
GGGC
SEQ ID NO: 3
VDDLMAKING GGC
linker
SEQ ID NO: 4
CGGG
AsTRIO antigenic epitope
SEQ ID NO: 5
AANLRDKEN
SEQ ID NO: 6
AANLRDKENG GGC

1. An immunogen comprising:
an immunogenic carrier comprising a Qβ bacteriophage virus-like particle (VLP); and
an antigenic Anopheles spp. TRIO peptide linked to the immunogenic carrier.

2. The immunogen of claim 1, wherein the antigenic Anopheles spp. TRIO peptide comprises VDDLMAKFN (SEQ ID NO:1) or a structurally similar variant thereof.

3. The immunogen of claim 1, wherein the antigenic Anopheles spp. TRIO peptide comprises AANLRDKFN (SEQ ID NO:5) or a structurally similar variant thereof.

4. The immunogen of claim 1, wherein the immunogenic carrier is linked to the TRIO peptide through a succinimidyl-6-[β-maleimidopropionamido]hexanoate (SMPH) cross-linker molecule.

5. The immunogen of claim 1, further comprising an antigenic malaria circumsporozoite protein (CSP) peptide or a structurally similar variant thereof.

6. The immunogen of claim 5, wherein the TRIO peptide and the antigenic CSP peptide are displayed on a single VLP.

7. A composition comprising the immunogen of claim 1.

8. The composition of claim 7, comprising:
a first population of VLPs displaying an antigenic Anopheles TRIO peptide comprising SEQ ID NO:1, SEQ ID NO:5, or a structurally similar variant of either SEQ ID NO:1 or SEQ ID NO:5; and
a second population of VLPs displaying an antigenic CSP peptide or a structurally similar variant thereof.

9. The composition of claim 7, comprising:
a first population of VLPs displaying an antigenic Anopheles TRIO peptide comprising SEQ ID NO:1 or a structurally similar variant thereof; and
a second population of VLPs displaying an antigenic Anopheles TRIO peptide comprising SEQ ID NO:5 or a structurally similar variant thereof.

10. The composition of claim 7, further comprising an adjuvant.

11. The composition of claim 10, wherein the adjuvant comprises a CpG oligonucleotide.

12. A method of treating malaria in an individual, the method comprising administering a therapeutically effective amount of a composition to the individual, the composition comprising an immunogen, the immunogen comprising:
an immunogenic carrier; and
an antigenic Anopheles spp. TRIO peptide linked to the immunogenic carrier.

13. The method of claim 12, wherein the antigenic Anopheles spp. TRIO peptide comprise SEQ ID NO:1 or a structurally similar variant thereof.

14. The method of claim 12, wherein the antigenic Anopheles spp. TRIO peptide comprise SEQ ID NO:5 or a structurally similar variant thereof.

15. The method of claim 12, wherein the method further comprises administering to the individual at least one additional therapeutic agent for treating malaria.

16. The method of claim 15, wherein the additional therapeutic agent for treating malaria comprises a pre-erythrocytic malaria vaccine.

17. The method of claim 16, wherein the pre-erythrocytic malaria vaccine comprises an L9 VLP vaccine, an RTS,S/AS01 vaccine, an R21 vaccine, or a PfSPZ vaccine.

18. The method of claim 12, wherein the immunogen further comprises an antigenic malaria circumsporozoite protein (CSP) peptide or a structurally similar variant thereof.

19. The method of claim 18, wherein the TRIO peptide and the antigenic CSP peptide are linked to a single carrier.

20. The method of claim 12, wherein the composition further comprises:
a second population of immunogens comprising:
a second population of immunogenic carriers; and
an immunogenic CSP peptide linked to the second population of immunogenic carriers.

21. The method of claim 12, wherein the immunogenic carrier comprises a Qβ virus-like particle (VLP).

22. A nucleic acid encoding the immunogen of claim 1.

23. An expression vector comprising the nucleic acid of claim 22.

24. A host cell comprising the expression vector of claim 23.

25-34. (canceled)


SEQUENCE LISTING (6 sequences)

SEQ ID NO: 1 - protein, 9 aa, synthetic construct
VDDLMAKFN

SEQ ID NO: 2 - protein, 4 aa, synthetic construct
GGGC

SEQ ID NO: 3 - protein, 13 aa, synthetic construct
VDDLMAKFNGGGC

SEQ ID NO: 4 - protein, 4 aa, synthetic construct
CGGG

SEQ ID NO: 5 - protein, 9 aa, synthetic construct
AANLRDKFN

SEQ ID NO: 6 - protein, 13 aa, synthetic construct
AANLRDKFNGGGC